(19) United States
(12) Patent Application Publication
(10) Pub. No.: US 2014/0153702 A1
(43) Pub. Date: Jun. 5, 2014

(54) IMAGING SYSTEM RESPONSIVE TO SUBJECT SIZE AND POSITION

(71) Applicant: Varad N. Srivastava, Batesville, IN (US)

(72) Inventor: Varad N. Srivastava, Batesville, IN (US)

(21) Appl. No.: 13/705,726

(22) Filed: Dec. 5, 2012

(51) Int. Cl. A61B 6/00

(52) U.S. Cl. CPC A61B 6/58
USPC 378/205

(57) ABSTRACT

An imaging system comprises a presence sensing subsystem responsive to presence and absence of a subject, a controller comprising a processor, configured to a) receive information representative of the presence and absence of the subject from the presence sensing subsystem, and to b) produce an output related to a site of interest, and an imaging subsystem responsive to the output such that the imaging subsystem targets the site of interest.

### TECHNICAL FIELD

The subject matter described herein relates to an imaging system in which an imaging emitter is positioned relative to a subject in response to information from a presence sensing subsystem so that the emitter targets a site of diagnostic interest on the subject. In one example the emitter is an xray generator and the site of interest is a site to be xray imaged.

### BACKGROUND

A patient who requires xrays or other imaging might not be ambulatory and/or may not be able to exert effort to move himself or herself as requested by an imaging technician. In addition, not all patients are equal or even similar in size. Accordingly, it would be advantageous to provide an imaging system that senses patient size and position and causes an xray generator or other imaging emitter to move to a location consistent with imaging a site of interest on the patient.

### SUMMARY

An imaging system comprises a presence sensing subsystem responsive to presence and absence of a subject and a controller comprising a processor. The controller is configured to receive information representative of the presence and absence of the subject from the presence sensing subsystem and to produce an output related to a site of interest. The imaging system also includes an imaging subsystem responsive to the output such that the imaging subsystem targets the site of interest.

### BRIEF DESCRIPTION OF THE DRAWINGS

The foregoing and other features of the various embodiments of the imaging system described herein will become more apparent from the following detailed description and the accompanying drawings in which:

FIG. 1 is a schematic plan view showing outlines of an adult patient and a child patient, each in a supine orientation on a hospital bed mattress, a pressure sensing overlay resting on the mattress underneath the patient outlines, and a ceiling mounted xray generator.

FIG. 2 is a side elevation view of the bed of FIG. 1 without the patients and also showing an xray generator mounted to a frame of the bed.

FIG. 3 is a block diagram of an imaging system comprising a presence sensing subsystem, a controller, a user interface, and an imaging subsystem in which the imaging subsystem is moveable to a location suitable for imaging a target site of interest on the patient in response to an output from the presence sensing subsystem.

FIG. 4 is a schematic view of a user interface.

### DETAILED DESCRIPTION

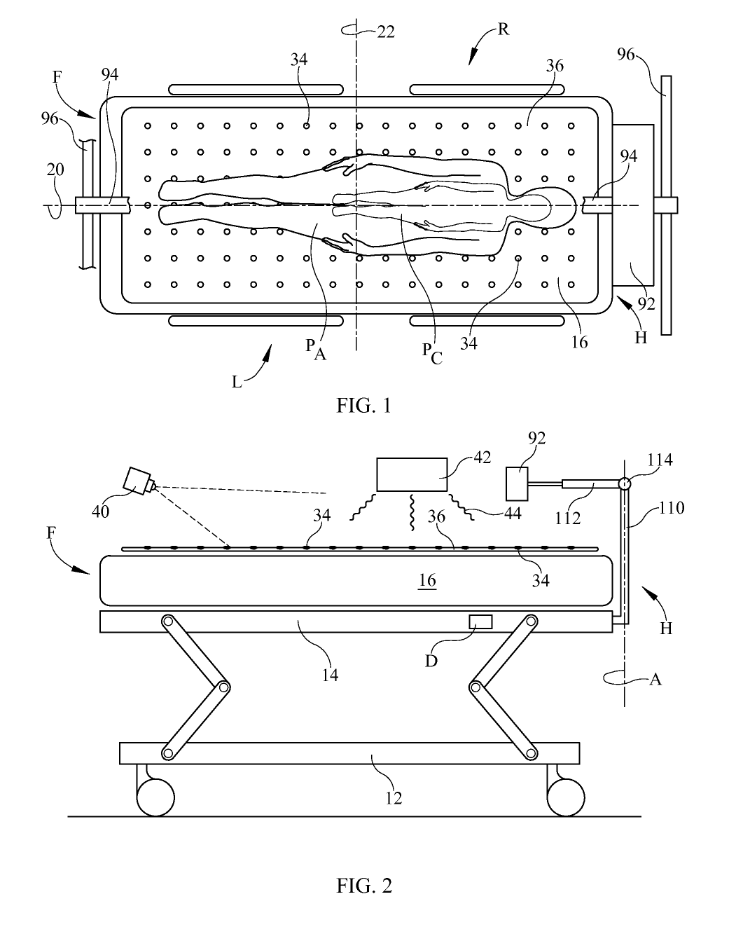

FIGS. 1-2 show a hospital bed 10 extending longitudinally from a head end H to a foot end F and laterally from a left side L to a right side R. The bed includes a frame 12 having a mattress support deck 14 and a mattress 16 resting on the deck. Longitudinally and laterally extending centerlines 20, 22 are illustrated for reference and for indicating the longitudinal and lateral directions. A patient or subject, such as an adult patient P_{A} or a child patient P_{c}, rests on the bed. The illustrated patients are each shown in a supine orientation.

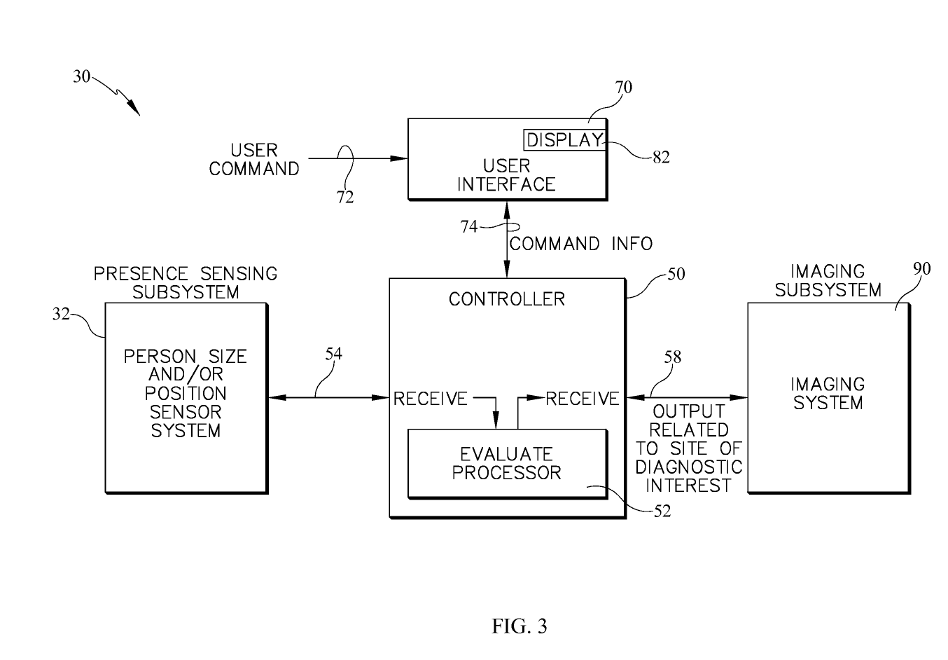

Referring additionally to FIG. 3, an imaging system 30 includes a presence sensing subsystem 32 to reveal an estimated spatial location of the subject and of his or her externally perceivable anatomical features such as head, neck, torso, thighs, calves, feet, arms and legs. The spatial location of the subject as a whole and/or of the subject's anatomical features depends on parameters such as the subject's size (e.g. height, width), position relative to centerlines 20, 22, posture (e.g. stretched out or curled in a fetal position), and orientation (supine, prone, lying on his/her left or right side). In the interest of simplicity, and unless the context indicates otherwise, references herein to a subject's location should be understood to include not only the location of the subject as a whole but also the locations of externally perceivable body parts and anatomical features. In one embodiment the presence sensing subsystem is a pressure sensing subsystem comprising an array of discrete pressure sensors 34. In the illustrated example the sensors are provided on an overlay 36 placed atop the mattress. In another example the array is embedded in the mattress. In yet another example the array is beneath the mattress, for example an underlay similar to overlay 36 residing between mattress 16 and deck 14 or an array of sensors such as sensors 34 integrated into the deck. The resolution with which the subject's spatial location can be determined depends on the spacing between the sensors. An algorithm can be used to carry out interpolations to improve the estimate of the subject's location.

In another embodiment the presence sensing subsystem is a pressure sensing subsystem comprising a pressure sensitive film which provides higher resolution information concerning the subject's spatial location than is possible with an array of discrete sensors. In yet another embodiment the presence sensing subsystem is a camera 40 positioned above the bed and sensitive to a portion of the electromagnetic spectrum such as visible light or infrared radiation. In still another embodiment the presence sensing subsystem is an acoustic detection system 42 that emits acoustic signals 44 toward the subject and mattress from above and senses the return signals reflected by the subject or the mattress to determine or enable a determination of the presence or absence of the subject. For example a signal with a relatively short transit time would indicate that it had reflected from the subject (and therefore from a spatial location where the subject is present) whereas a signal with a relatively longer transit time would indicate that it had reflected from the mattress (and therefore from a spatial location where the subject is absent). Intermediate transit times can indicate the presence of a thin portion of the subject's body, for example a hand resting palm down on the mattress.

The reader should appreciate that the presence sensing subsystem detects both presence and absence of the subject. Accordingly, the presence sensing subsystem may be thought of as a “presence and absence sensing subsystem”. In the interest of simplicity this specification will frequently use the phrase “presence sensing subsystem” as an abbreviated way to refer to a “presence and absence sensing subsystem”. Other similar phrases using “presence” or variations thereof likewise refer to the alternative, mutually exclusive state of absence.

As explained in the foregoing paragraphs, optional embodiments of the the presence sensing subsystem include a pressure sensing subsystem employing pressure sensing technology, an electromagnetic sensing subsystem employing electromagnetic technology and an acoustic sensing subsystem employing acoustic technology. The use of other sensing subsystems and technologies may also be satisfactory. Irrespective of the particular technology employed, an interpolating algorithm may be used to develop a more definite, higher resolution estimate of the subject's location, and may be particularly useful when patient location is determined as a function of discrete sensors spaced widely enough apart that the resulting estimate of subject location would otherwise be undesirably granular.

The imaging system also includes a controller 50 comprising a processor 52. Other elements of the controller may include components such as a power supply, a voltage regulator and communication modules for sending or receiving information. As illustrated, only a single processor is used however processing (e.g. computation) may be distributed among two or more processors. In one example the illustrated processor receives presence and absence information from presence sensing subsystem 32, conducts basic data validition and fault checking operations, and transfers the data to a remote server for further processing. The processor, whether distributed or not, receives information 54 representative of the presence and absence of the subject from the presence sensing subsystem, evaluates the location and spatial distribution of the subject as a function of the received information, and produces an output 58 related to a site of diagnostic interest.

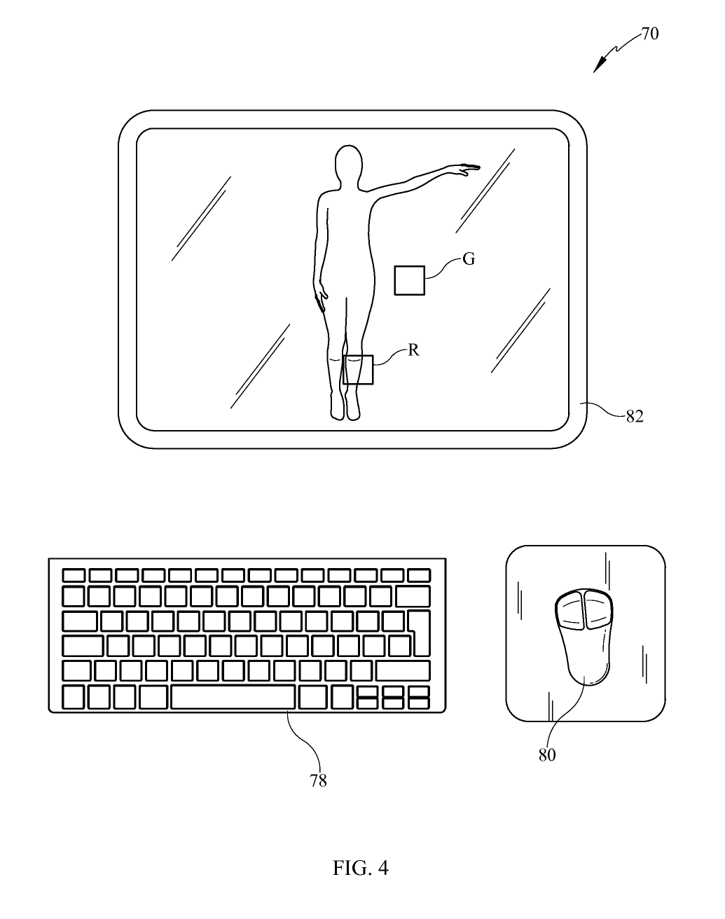

Referring additionally to FIG. 4, the imaging system also includes a user interface 70 configured to receive a command 72 from a user and to communicate command information to controller 50. In one example the user interface includes a keyboard 78 and mouse 80 and a display monitor 82. The monitor component of the user interface is configured to display information representative of the location and spatial distribution of the subject, for example a plan view of the subject, and to display an indication of a user selected target site of diagnostic interest. The user uses the keyboard and mouse to issue a command or commands 72, and observes the monitor to assess the effect of those commands having been carried out. For example, the monitor may show an outline of the subject based on information 54 obtained from the presence sensing subsystem and a red-line target box R to reveal a user selected target site of diagnostic interest. The user employs the keyboard and/or mouse to adjust the size of box R and to drag the box over a site of diagnostic interest on the subject's body. As depicted in FIG. 4, the subject's left knee is the site of diagnostic interest.

The imaging system also includes an imaging subsystem 90. One example of an imaging subsystem is an x-ray subsystem comprising an xray generator 92 and a digital or nondigital film, not illustrated, on the opposite side of the subject. The illustrated xray generator is mounted on and longitudinally translatable along a longitudinally extending rail 94 which itself is laterally translatable, e.g. along laterally extending rails 96 secured to the ceiling of the room. Consequently the xray generator can be positioned at a desired location above the subject. The imaging subsystem is responsive to controller output 58 such that the imaging subsystem targets the site of interest. In one embodiment output 58 causes the xray generator to translate longitudinally and/or laterally (by virtue of rail 94 translating along rails 96) until it is over the site of interest. A green-line box G on display monitor 82 changes position to indicate the actual position of the generator, allowing the technician to confirm that the generator is in proper position to image the site of interest outlined by red box R. The technician can then operate the xray generator to produce an xray image of the site on the xray film.

In the foregoing example the imaging subsystem is an xray subsystem in which the emitter (xray generator) and detector (digital or nondigital film) are on opposite sides of the subject. Other types of imaging subsystems based on other technologies such as sonography and/or in which the emitter and detector are on the same side of the subject may also be used. In addition, alternatives to the ceiling mounted arrangement described above can be employed. For example the xray generator (or other emitter) could be a stand-alone unit that the caregiver staff moves into position near the bed or attaches to the bed on an as-needed basis. The bed could include one or more docking ports D to which the stand alone unit is docked. Such ports may be useful for reasons such as providing an electrical power connection and providing for data connectivity. In another example, depicted in FIG. 2, the xray generator 92 or other emitter is permanently or nonpermanently mounted to the bed by way of a stanchion 110 and a support arm 112. The support arm is connected to the stanchion at a joint 114 which allows the arm to pivot relative to the stanchion about at least a vertical axis A. Arm 112 is a telescoping arm so that the generator can be moved away from or toward joint 114.

In one mode of operation a user enters a command 72 indicating which portion of the subject's body the user wishes to image, for example the subject's left knee. The processor uses information 54 from presence sensing subsystem 32 to gauge the position of the knee and produces output 58 to cause xray generator 92 to move to a preliminary location in the vicinity of the knee. If necessary the user then enters additional commands via mouse 80 and/or keyboard 78 to fine-tune the location of xray generator 92. In another mode of operation the preliminary positioning of the xray generator is dispensed with and the positioning is entirely under the control of the user via. the mouse and/or keyboard.

FIG. 4 shows communication paths by which information and data (e.g. controller information 54, controller output 58, command information 74) are conveyed and shared amongst the various subsystems. The communication paths may be physical connections that rely on, for example, wires or optical cables, or may be wireless. The reader should also appreciate that the user interface may be in close proximity to the bed, or may be remote from the bed, for example in another room or another building. In order to properly position the xray generator the controller accounts for the location of the subject as indicated by the presence sensing subsystem and the location of the xray generator or other emitter. In other words the controller relates the reference frame (e.g. spatial coordinates) of the subject to the reference frame (e.g. spatial coordinates) of the xray generator. If the xray generator is mounted on the bed as seen in FIG. 2 or is docked to a docking port D (also seen in FIG. 2) the controller can employ a coordinate system common to both the subject and the xray generator. In other arrangements where the location of the xray generator relative to the sensor elements (e.g. sensors 34) is subject to variation and lack of repeatability, an algorithm to translate between coordinate systems can be employed.

Although this disclosure refers to specific embodiments, it will be understood by those skilled in the art that various changes in form and detail may be made without departing from the subject matter set forth in the accompanying claims.

I claim:

1. An imaging system comprising:
a presence sensing subsystem responsive to presence and absence of a subject;
a controller comprising a processor, the controller being configured to
a) receive information representative of the presence and absence of the subject from the presence sensing subsystem, and
b) produce an output related to a site of interest; and
an imaging subsystem responsive to the output such that the imaging subsystem targets the site of interest.

2. The imaging system of claim 1 comprising a user interface configured to receive a command from a user and to communicate command information to the controller.

3. The imaging system of claim 2 wherein the user interface is also configured to display information representative of the location and spatial distribution of the subject.

4. The imaging system of claim 3 wherein the user interface is configured to also display an indication of a user selected target site.

5. The imaging system of claim 3 wherein the user interface is configured to display the representative information as a plan view of the subject.

6. The imaging system of claim 5 wherein the user interface is configured to also display an indication of a user selected target site.

7. The imaging system of claim 1 wherein the processor is distributed.

8. The imaging system of claim 1 wherein the presence sensing subsystem employs pressure sensing technology.

9. The imaging system of claim 1 wherein the presence sensing subsystem employs electromagnetic technology.

10. The imaging system of claim 1 wherein the presence sensing subsystem employs acoustic technology.

11. The imaging system of claim 1 wherein the presence sensing subsystem is a pressure sensing subsystem.

12. The imaging system of claim 1 wherein the presence sensing subsystem is an electromagnetic sensing subsystem.

13. The imaging system of claim 1 wherein the presence sensing subsystem is an acoustic sensing subsystem.

14. The imaging system of claim 1 wherein the imaging subsystem is an x-ray subsystem.
